(19) United States
(12) Patent Application Publication
(10) Pub. No.: US 2022/0071704 A1
(43) Pub. Date: Mar. 10, 2022

(54) VALVULOPLASTY TREATMENT SYSTEM AND METHOD

(71) Applicant: BOLT MEDICAL, INC., Carlsbad, CA (US)

(72) Inventor: Khoi Le, Lake Forest, CA (US)

(21) Appl. No.: 17/463,713

(22) Filed: Sep. 1, 2021

Related U.S. Application Data
(60) Provisional application No. 63/076,035, filed on Sep. 9, 2020.

(51) Int. Cl. A61B 18/24; A61B 18/26

(52) U.S. Cl. CPC A61B 18/24; A61B 2018/00369; A61B 18/26

(57) ABSTRACT

A catheter system (100) for treating a vascular lesion (106) within or adjacent to a heart valve (108) within a body (107) of a patient (109), includes an energy source (124), and a plurality of spaced apart treatment devices (143). The energy source (124) generates energy. Each treatment device (143) includes (i) a balloon (104) that is positionable substantially adjacent to the vascular lesion (106), the balloon (104) having a balloon wall (130) that defines a balloon interior (146), the balloon (104) being configured to retain a balloon fluid (132) within the balloon interior (146); and (ii) at least one of a plurality of energy guides (122A) that receive energy from the energy source (124) so that plasma (134) is formed in the balloon fluid (132) within the balloon interior (146).

### RELATED APPLICATION

This application claims priority on U.S. Provisional Application Ser. No. 63/076,035, filed on Sep. 9, 2020. To the extent permitted, the contents of U.S. Provisional Application Ser. No. 63/076,035 are incorporated in their entirety herein by reference.

### BACKGROUND

Vascular lesions, such as calcium deposits, within and adjacent to heart valves in the body can be associated with an increased risk for major adverse events, such as myocardial infarction, embolism, deep vein thrombosis, stroke, and the like. Severe vascular lesions, such as severely calcified vascular lesions, can be difficult to treat and achieve patency for a physician in a clinical setting.

The tricuspid valve, also known as the right atrioventricular valve, includes three leaflets which open and close in unison when the valve is functioning properly. The tricuspid valve functions as a one-way valve that opens during ventricular diastole, allowing blood to flow from the right atrium into the right ventricle, and closes during ventricular systole to prevent regurgitation of blood from the right ventricle back into the right atrium. The back flow of blood, also known as regression or tricuspid regurgitation, can result in increased ventricular preload because the blood refluxed back into the atrium is added to the volume of blood that must be pumped back into the ventricle during the next cycle of ventricular diastole. Increased right ventricular preload over a prolonged period of time may lead to right ventricular enlargement (dilatation), which can progress to right heart failure if left uncorrected.

A calcium deposit on the tricuspid valve, known as valvular stenosis, can form adjacent to a valve wall of the tricuspid valve and/or on or between the leaflets of the tricuspid valve. Valvular stenosis can prevent the leaflets from opening and closing completely, which can, in turn, result in the undesired tricuspid regurgitation. Over time, such calcium deposits can cause the leaflets to become less mobile and ultimately prevent the heart from supplying enough blood to the rest of the body.

Certain methods are currently available which attempt to address valvular stenosis, but such methods have not been altogether satisfactory. One such method includes using a standard balloon valvuloplasty catheter. Unfortunately, this type of catheter typically does not have enough strength to sufficiently disrupt the calcium deposit between the leaflets or at the base of the leaflets. Another such method includes artificial tricuspid valve replacement, which can be used to restore functionality of the tricuspid valve. However, this procedure is highly invasive and extremely expensive. In still another such method, a valvular stent can be placed between the leaflets to bypass the leaflets. This procedure is relatively costly and results have found that the pressure gradient does not appreciably improve.

Thus, there is an ongoing desire to develop improved methodologies for valvuloplasty in order to more effectively and efficiently break up calcium deposits adjacent to the valve wall of the tricuspid valve and/or between the leaflets of the tricuspid valve. It is also desired that such improved methodologies work effectively to address not only valvular stenosis related to the tricuspid valve, but also calcification on other heart valves, such as mitral valve stenosis within the mitral valve and aorta valve stenosis within the aorta valve.

### SUMMARY

The present invention is directed toward a catheter system for placement within a heart valve. The catheter system can be used for treating a vascular lesion within or adjacent to the heart valve within a body of a patient. In various embodiments, the catheter system includes an energy source, and a plurality of spaced apart treatment devices. The energy source generates energy. Each treatment device includes (i) a balloon that is positionable substantially adjacent to the vascular lesion, the balloon having a balloon wall that defines a balloon interior, the balloon being configured to retain a balloon fluid within the balloon interior; and (ii) at least one of a plurality of energy guides that receive energy from the energy source so that plasma is formed in the balloon fluid within the balloon interior.

In certain embodiments, at least one of the balloons has a drug eluting coating.

In some applications, the heart valve includes a valve wall, and the balloon of each of the treatment devices is configured to be positioned adjacent to the valve wall.

In certain embodiments, each treatment device further includes an inflation tube, and the balloon fluid is transmitted into the balloon interior via the inflation tube. In some such embodiments, the balloon of each of the treatment devices includes a balloon proximal end that is coupled to the inflation tube.

In some embodiments, the catheter system further includes a plurality of plasma generators, with one corresponding plasma generator of the plurality of plasma generators being positioned near a guide distal end of each of the plurality of energy guides, wherein each plasma generator is configured to generate the plasma in the balloon fluid within the balloon interior.

In certain embodiments, the plasma formation causes rapid bubble formation and imparts pressure waves upon the balloon wall of each of the balloons adjacent to the vascular lesion.

In some embodiments, the energy source generates pulses of energy that are guided along each of the plurality of energy guides into the balloon interior of each balloon to induce the plasma formation in the balloon fluid within the balloon interior of each of the balloons.

In certain embodiments, the energy source is a laser source that provides pulses of laser energy.

In some embodiments, at least one of the plurality of energy guides includes an optical fiber.

In one embodiment, the energy source is a high voltage energy source that provides pulses of high voltage.

In one embodiment, at least one of the plurality of energy guides includes an electrode pair including spaced apart electrodes that extend into the balloon interior; and pulses of high voltage from the energy source are applied to the electrodes and form an electrical arc across the electrodes.

In certain embodiments, the catheter system further includes an inner shaft, wherein a device proximal end of each of the plurality of spaced apart treatment devices is coupled to the inner shaft.

In some such embodiments, the catheter system further includes a plurality of device couplers. In such embodiments, the device proximal end of each of the plurality of spaced apart treatment devices is coupled to the inner shaft via one of the plurality of device couplers.

In certain such embodiments, each treatment device further includes an inflation tube, the balloon fluid being transmittable into the balloon interior via the inflation tube, the inner shaft including an inner shaft body that defines a plurality of inner shaft lumens, and the inflation tube of the treatment devices each being coupled to one of the plurality of inner shaft lumens.

In some embodiments, the catheter system further includes a guidewire that is configured to guide movement of the plurality of treatment devices so that the balloon of each of the treatment devices is positioned substantially adjacent to the vascular lesion. In such embodiments, the catheter system can include three spaced apart treatment devices that are spaced apart approximately 120 degrees from one another about the guidewire.

In certain embodiments, the catheter system further includes a deployment collet that is fixedly secured to the guidewire such that movement of the guidewire causes corresponding movement of the deployment collet.

In some embodiments, the guidewire is positioned to extend through the heart valve and the inner shaft is configured to be fixed in position relative to the heart valve during use of the catheter system. In such embodiments, pulling back on the guidewire causes the treatment devices to fan outwardly so that the balloon of each treatment device moves toward the vascular lesion.

In certain embodiments, a device distal end of each of the treatment devices is coupled to the deployment collet, and each treatment device further includes an inner tube that is coupled to the deployment collet at the device distal end of each of the treatment devices.

In some embodiments, each treatment device further includes a guide positioner that is positioned about the inner tube, the guide positioner being configured to control a position of the at least one of the plurality of energy guides that is included within the treatment device.

The present invention is further directed toward a method for treating a vascular lesion within or adjacent to a heart valve utilizing the catheter system as described above.

The present invention is also directed toward a method for treating a vascular lesion within or adjacent to a heart valve within a body of a patient, the method comprising the steps of generating energy with an energy source; receiving energy from the energy source with a plurality of energy guides; and positioning a plurality of treatment devices spaced apart from one another, each treatment device including (i) a balloon that is positionable substantially adjacent to the vascular lesion, the balloon having a balloon wall that defines a balloon interior, the balloon being configured to retain a balloon fluid within the balloon interior; and (ii) at least one of the plurality of energy guides that receive the energy from the energy source so that plasma is formed in the balloon fluid within the balloon interior.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic cross-sectional view of an embodiment of a catheter system in accordance with various embodiments herein, the catheter system including a valvuloplasty treatment system having features of the present invention;

FIG. 2 is a simplified perspective view of a portion of an embodiment of the valvuloplasty treatment system;

FIG. 3 is a simplified perspective view of a portion of a multi-lumen outer shaft that can form part of the valvuloplasty treatment system illustrated in FIG. 2;

FIG. 4 is a simplified perspective view of an external cap that can form part of the valvuloplasty treatment system illustrated in FIG. 2;

FIG. 5 is a simplified perspective view of a portion of a movable multi-lumen inner shaft that can form part of the valvuloplasty treatment system illustrated in FIG. 2;

FIG. 6 is a simplified perspective view of a deployment collet that can form part of the valvuloplasty treatment system illustrated in FIG. 2;

FIG. 7A is a simplified perspective view of a portion of the multi-lumen outer shaft, the movable multi-lumen inner shaft, and a treatment device that can form a part of the valvuloplasty treatment system illustrated in FIG. 2, the treatment device being shown in a first (retracted) position;

FIG. 7B is another simplified perspective view of a portion of the multi-lumen outer shaft, the movable multi-lumen inner shaft, and the treatment device illustrated in FIG. 7A, the treatment device being shown in a second (extended) position;

FIG. 7C is still another simplified perspective view of a portion of the treatment device illustrated in FIG. 7A;

FIG. 7D is yet another simplified perspective view of a portion of the treatment device illustrated in FIG. 7A;

FIG. 8 is a simplified perspective view of a portion of an energy guide usable as part of the treatment device illustrated in FIG. 7A;

FIG. 9A is a simplified perspective view of an embodiment of a plasma target ring usable as part of the treatment device illustrated in FIG. 7A;

FIG. 9B is a simplified end view of another embodiment of the plasma target ring illustrated in FIG. 9A, and a portion of an inner tube and guide positioner that are usable as part of the treatment device; and

FIG. 10 is a flowchart that illustrates one representative application of a use of the valvuloplasty treatment system as part of the catheter system.

While embodiments of the present invention are susceptible to various modifications and alternative forms, specifics thereof have been shown by way of example and drawings, and are described in detail herein. It is understood, however, that the scope herein is not limited to the particular embodiments described. On the contrary, the intention is to cover modifications, equivalents, and alternatives falling within the spirit and scope herein.

### DESCRIPTION

The catheter systems and related methods disclosed herein are configured to incorporate improved methodologies for valvuloplasty in order to more effectively and efficiently break up any calcified vascular lesions that may have developed on and/or within the heart valves over time. More particularly, the catheter systems and related methods generally include a valvuloplasty treatment system that incorporates the use of a plurality of spaced apart, individual treatment devices, with each treatment device incorporating and/or encompassing a balloon catheter, that are moved so as to be positioned within and/or adjacent to the heart valve. The treatment devices are then anchored in specific locations so that energy can be directed to the precise locations necessary at the heart valve, such as adjacent to the valve wall and/or on or between adjacent leaflets within the heart valve, in order to break up the calcified vascular lesions. While such methodologies are often described herein as being useful for treatment of valvular stenosis in relation to the tricuspid valve, it is appreciated that such methodologies are also useful in treatment of calcium deposits on other heart valves, such as for mitral valve stenosis within the mitral valve and for aorta valve stenosis within the aorta valve.

As used herein, the terms “intravascular lesion” and “vascular lesion” are used interchangeably unless otherwise noted. As such, the intravascular lesions and/or the vascular lesions are sometimes referred to herein simply as “lesions”.

Those of ordinary skill in the art will realize that the following detailed description of the present invention is illustrative only and is not intended to be in any way limiting. Other embodiments of the present invention will readily suggest themselves to such skilled persons having the benefit of this disclosure. Other methods of delivering energy to the lesion can be utilized, including, but not limited to electric current induced plasma generation. Reference will now be made in detail to implementations of the present invention as illustrated in the accompanying drawings. The same or similar nomenclature and/or reference indicators will be used throughout the drawings and the following detailed description to refer to the same or like parts.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it is appreciated that such a development effort might be complex and time-consuming, but would nevertheless be a routine undertaking of engineering for those of ordinary skill in the art having the benefit of this disclosure.

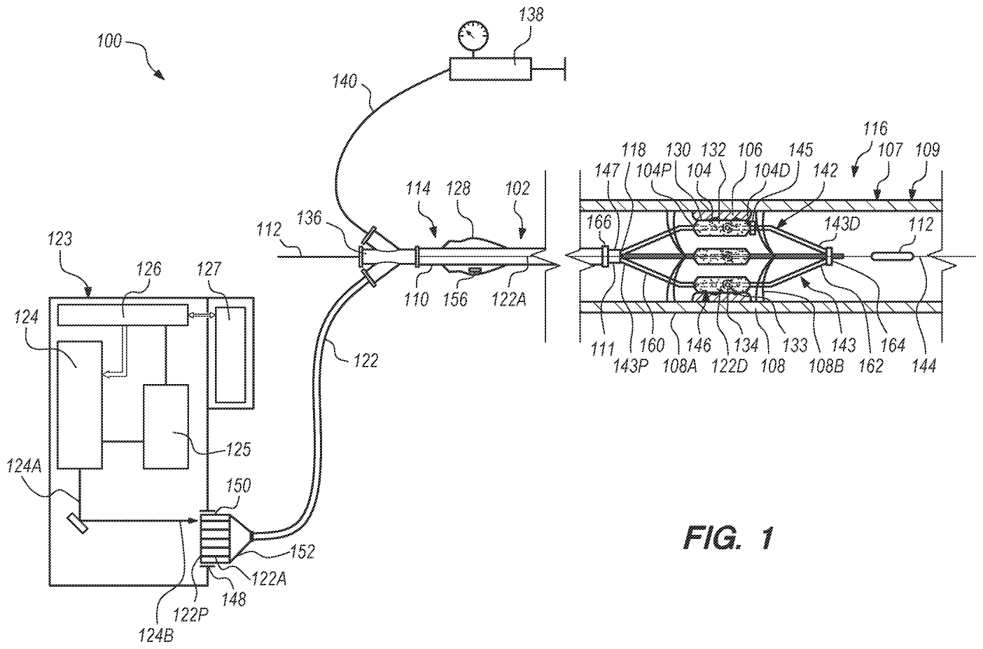

It is appreciated that the catheter systems disclosed herein can include many different forms. Referring now to FIG. 1, a schematic cross-sectional view is shown of a catheter system 100 in accordance with various embodiments herein. The catheter system 100 is suitable for imparting pressure to induce fractures in one or more vascular lesions adjacent to the valve wall and/or on or between adjacent leaflets within the tricuspid valve (or other heart valves). In the embodiment illustrated in FIG. 1, the catheter system 100 can include one or more of a valvuloplasty treatment system 142 (also referred to herein more simply as a “treatment system”) that incorporates, encompasses and/or utilizes a catheter 102, an energy guide bundle 122 (e.g., a light guide bundle) including one or more energy guides 122A (e.g., light guides), a source manifold 136, a fluid pump 138, a system console 123 including one or more of an energy source 124 (e.g., a light source), a power source 125, a system controller 126, and a graphic user interface 127 (a “GUI”), and a handle assembly 128. The treatment system 142 and/or the catheter 102 includes spaced apart, individual treatment devices 143 to be used adjacent to a valve wall 108A and/or on or between adjacent leaflets 1088 within a heart valve 108, e.g., the tricuspid valve, at a treatment site 106. Alternatively, the catheter system 100 can have more components or fewer components than those specifically illustrated and described in relation to FIG. 1.

The treatment system 142 and/or the catheter 102 is configured to move to the treatment site 106 within or adjacent to the heart valve 108 within a body 107 of a patient 109. The treatment site 106 can include one or more vascular lesions such as calcified vascular lesions, for example. Additionally, or in the alternative, the treatment site 106 can include vascular lesions such as fibrous vascular lesions.

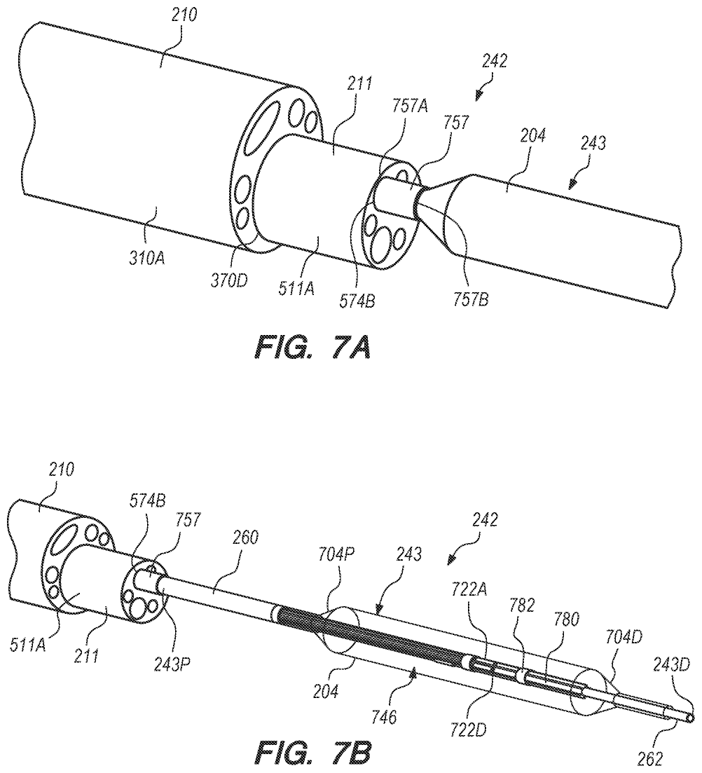

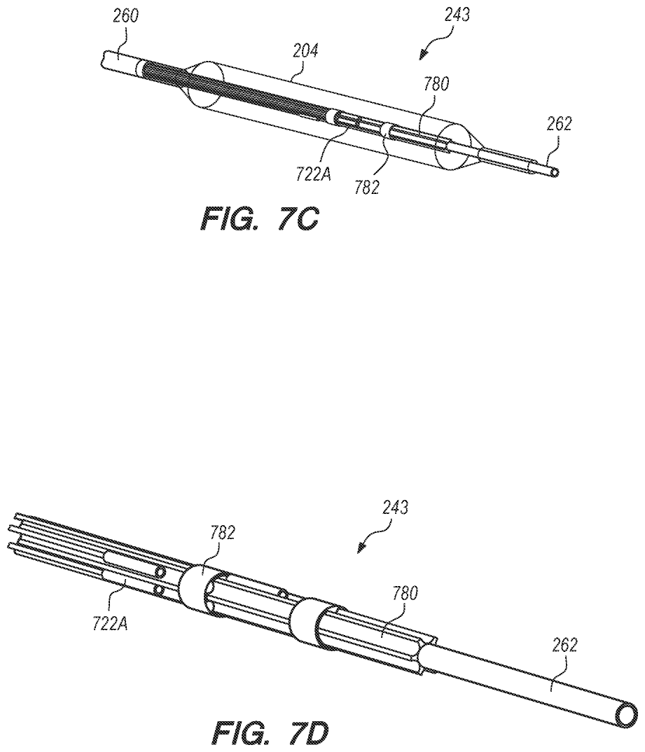

The treatment system 142 and/or the catheter 102 can include a multi-lumen outer shaft 110 (also referred to herein simply as an “outer shaft”), a movable multi-lumen inner shaft 111 (also referred to herein simply as an “inner shaft”) that is movably positioned within the outer shaft 110, and a plurality of spaced apart, individual treatment devices 143 that are coupled to the inner shaft 111, such as with a device coupler 757 (illustrated in FIG. 7A). For example, in one embodiment, the treatment system 142 and/or the catheter 102 includes three individual treatment devices 143. Alternatively, the treatment system 142 and/or the catheter 102 can include more than three individual treatment devices 143 or only two treatment devices 143.

The treatment system 142 is configured to impart pressure waves and/or fracture forces within each of the individual treatment devices 143 adjacent to the valve wall 108A and/or on or between adjacent leaflets 1088 within the heart valve 108 at the treatment site 106. Such pressure waves and/or fracture forces are utilized to break apart the vascular lesions that are located at the treatment site 106. It is appreciated that the treatment system 142 can also be utilized such that fewer than all of the individual treatment devices 143 are being utilized at any given time, for example, such that only two of three individual treatment devices 143 are being used at a given time.

As illustrated in FIG. 1, each individual treatment device 143 can include an inflation tube 160 that is movably coupled to the inner shaft 111 at a device proximal end 143P, an inner tube 162 that is coupled to a deployment collet 164 at a device distal end 143D, an inflatable balloon 104 (sometimes referred to herein simply as a “balloon”), and one or more of the energy guides 122A that are included within the energy guide bundle 122. The individual treatment devices 143 are configured to be spaced apart from one another. With such design, during use of the catheter system 100, the balloon 104 of each treatment device 143 is spaced apart from the balloon 104 of each of the other treatment devices 143.

The outer shaft 110 can extend from a proximal portion 114 of the catheter system 100 to a distal portion 116 of the catheter system 100. During deployment of the treatment system 142, the outer shaft 110 is initially inserted into the body 107 of the patient 109, such as via an artery or other suitable blood vessel, so that the outer shaft 110 is positioned a predetermined distance away from the heart valve 108, i.e. away from the treatment site 106 within or adjacent to the heart valve 108. In some non-exclusive applications, the outer shaft 110 can be positioned and parked at a predetermined distance of approximately 10-15 millimeters (mm) away from the heart valve 108. Alternatively, the outer shaft 110 can be positioned greater than 15 mm or less than 10 mm away from the heart valve 108.

In certain embodiments, the treatment system 142 can further include an external cap 166 that is configured to fit over a shaft distal end of the outer shaft 110. In such embodiments, the external cap 166 can further enhance and/or stabilize movement between the inner shaft 111 and the outer shaft 110. Alternatively, the treatment system 142 can be designed without the external cap 166.

As noted, the inner shaft 111 is movably positioned within the outer shaft 110. The inner shaft 111 can include a longitudinal axis 144. The inner shaft 110 can also include a guidewire lumen 118 which is configured to move over a guidewire 112 that is configured to guide movement of the inner shaft 111 and, thus, the treatment devices 143 into and through the heart valve 108. As shown, the deployment collet 164 can be fixedly coupled to the guidewire 112. During deployment of the treatment system 142, after the outer shaft 110 has been positioned as noted above, the inner shaft 111 with the guidewire 112 is inserted through a working channel of the outer shaft 110 and advanced past the leaflets 1088 of the heart valve 108 and into the right heart atrium of the heart.

The inner shaft 111 can be inserted such that the treatment devices 143 are positioned so that the leaflets 1088 of the heart valve 108 are close to a middle of the balloon 104 of each treatment device 143. More particularly, in various applications, the inner shaft 111 can be inserted such that the middle of each balloon 104 is positioned just past the leaflets 1088 of the heart valve 108. Subsequently, the guidewire 112 can be pulled back slightly, while maintaining the position of the inner shaft 111 and the device proximal end 143P of each of the treatment devices 143, such that the treatment devices 143 fan outwardly so that the middle of each balloon 104 is positioned substantially adjacent to the treatment site 106 on or adjacent to the leaflets 1088 of the heart valve 108. With such positioning, as described in greater detail herein below, energy from the energy source 124 can be guided through the energy guides 122A and directed and focused in a generally outward direction from the balloon 104 of each treatment device 143 and between the leaflets 1088 of the heart valve 108. It is further appreciated that the treatment devices 143, and thus the balloons 104, can be rotated as necessary such that the treatment devices 143 are properly lined up so that the energy from the energy source 124 can be more precisely directed and focused between the leaflets 1088 of the heart valve 108. With this design, the individual treatment devices 143 can be effectively utilized to break apart the vascular lesions adjacent to the valve wall 108A and/or on or between adjacent leaflets 1088 within the heart valve 108 at the treatment site 106.

In some embodiments, the treatment system 142 can include one or more filters 145 that are configured to capture and/or trap debris generated from the breaking up of the vascular lesions at the treatment site 106 to inhibit such debris from entering the blood stream. For example, in one such embodiment, a separate filter 145 can be coupled to each of the treatment devices 143.

In certain embodiments, the catheter system 100 and/or the treatment system 142 can further include an imaging system 147 (illustrated as a box in phantom), such as a complementary metal oxide semiconductor (CMOS) imaging system, that can be used to more accurately and precisely guide the positioning of the outer shaft 110, the inner shaft 111, and/or the individual treatment devices 143 within the body 107 of the patient 109.

In various embodiments, the balloon 104 of each treatment device 143 includes a balloon proximal end 104P that is coupled to the inflation tube 160, and a balloon distal end 104D that is coupled to the inner tube 162. Each balloon 104 can include a balloon wall 130 that defines a balloon interior 146, and can be inflated with a balloon fluid 132, e.g., via the inflation tube 160, to expand from a deflated configuration suitable for advancing the treatment system 142 and/or the treatment device 143 through a patient's vasculature, to an inflated configuration suitable for anchoring the treatment system 142 and/or the treatment device 143 in position relative to the treatment site 106. Stated in another manner, when the balloon 104 is in the inflated configuration, the balloon wall 130 of the balloon 104 is configured to be positioned substantially adjacent to the treatment site 106, i.e. to the vascular lesion(s).

The balloons 104 suitable for use in the catheter systems 100 include those that can be passed through the vasculature of a patient when in the deflated configuration. In some embodiments, the balloons 104 are made from silicone. In various embodiments, the balloons 104 are made from polydimethylsiloxane (PDMS), polyurethane, polymers such as PEBAX™ material available from Arkema, which has a location at King of Prussia, Pa., USA, nylon, and the like. In some embodiments, the balloons 104 can include those having diameters ranging from one millimeter (mm) to 25 mm in diameter. In certain embodiments, the balloons 104 can include those having diameters ranging from at least 1.5 mm to 14 mm in diameter. In some embodiments, the balloons 104 can include those having diameters ranging from at least one mm to five mm in diameter.

In some embodiments, the balloons 104 can include those having a length ranging from at least three mm to 300 mm. More particularly, in some embodiments, the balloons 104 can include those having a length ranging from at least eight mm to 200 mm. It is appreciated that balloons 104 of greater length can be positioned adjacent to larger treatment sites 106, and, thus, may be usable for imparting pressure onto and inducing fractures in larger vascular lesions or multiple vascular lesions at precise locations within the treatment site 106.

The balloons 104 can be inflated to inflation pressures of between approximately one atmosphere (atm) and 70 atm. In some embodiments, the balloons 104 can be inflated to inflation pressures of from at least 20 atm to 70 atm. In other embodiments, the balloons 104 can be inflated to inflation pressures of from at least six atm to 20 atm. In certain embodiments, the balloons 104 can be inflated to inflation pressures of from at least three atm to 20 atm. In various embodiments, the balloons 104 can be inflated to inflation pressures of from at least two atm to ten atm.

The balloons 104 can include those having various shapes, including, but not to be limited to, a conical shape, a square shape, a rectangular shape, a spherical shape, a conical/square shape, a conical/spherical shape, an extended spherical shape, an oval shape, a tapered shape, a bone shape, a stepped diameter shape, an offset shape, or a conical offset shape. In some embodiments, the balloons 104 can include a drug eluting coating or a drug eluting stent structure. The drug elution coating or drug eluting stent can include one or more therapeutic agents including anti-inflammatory agents, anti-neoplastic agents, anti-angiogenic agents, and the like.

The balloon fluid 132 can be a liquid or a gas. Exemplary balloon fluids 132 can include, but are not limited to one or more of water, saline, contrast medium, fluorocarbons, perfluorocarbons, gases, such as carbon dioxide, and the like. In some embodiments, the balloon fluids 132 described can be used as base inflation fluids. In some embodiments, the balloon fluids 132 include a mixture of saline to contrast medium in a volume ratio of 50:50. In other embodiments, the balloon fluids 132 include a mixture of saline to contrast medium in a volume ratio of 25:75. In still other embodiments, the balloon fluids 132 include a mixture of saline to contrast medium in a volume ratio of 75:25. The balloon fluids 132 can be tailored on the basis of composition, viscosity, and the like in order to manipulate the rate of travel of the pressure waves therein. In certain embodiments, the balloon fluids 132 are biocompatible. A volume of balloon fluid 132 can be tailored by the chosen energy source 124 and the type of balloon fluid 132 used.

In some embodiments, the contrast agents used in the contrast media can include, but are not to be limited to, iodine-based contrast agents, such as ionic or non-ionic iodine-based contrast agents. Some non-limiting examples of ionic iodine-based contrast agents include diatrizoate, metrizoate, iothalamate, and ioxaglate. Some non-limiting examples of non-ionic iodine-based contrast agents include iopamidol, iohexol, ioxilan, iopromide, iodixanol, and ioversol. In other embodiments, non-iodine based contrast agents can be used. Suitable non-iodine containing contrast agents can include gadolinium (III)-based contrast agents. Suitable fluorocarbon and perfluorocarbon agents can include, but are not to be limited to, agents such as the perfluorocarbon dodecafluoropentane (DDFP, C5F12).

The balloon fluids 132 can include those that include absorptive agents that can selectively absorb light in the ultraviolet region (e.g., at least ten nanometers (nm) to 400 nm), the visible region (e.g., at least 400 nm to 780 nm), or the near-infrared region (e.g., at least 780 nm to 2.5 μm) of the electromagnetic spectrum. Suitable absorptive agents can include those with absorption maxima along the spectrum from at least ten nm to 2.5 μm. Alternatively, the balloon fluids 132 can include those that include absorptive agents that can selectively absorb light in the mid-infrared region (e.g., at least 2.5 μm to 15 μm), or the far-infrared region (e.g., at least 15 μm to one mm) of the electromagnetic spectrum. In various embodiments, the absorptive agent can be those that have an absorption maximum matched with the emission maximum of the laser used in the catheter system 100. By way of non-limiting examples, various lasers described herein can include neodymium:yttrium-aluminum-garnet (Nd:YAG−emission maximum=1064 nm) lasers, holmium:YAG (Ho:YAG−emission maximum=2.1 μm) lasers, or erbium:YAG (Er:YAG−emission maximum=2.94 μm) lasers. In some embodiments, the absorptive agents can be water soluble. In other embodiments, the absorptive agents are not water soluble. In some embodiments, the absorptive agents used in the balloon fluids 132 can be tailored to match the peak emission of the energy source 124. Various energy sources 124 having emission wavelengths of at least ten nanometers to one millimeter are discussed elsewhere herein.

It is appreciated that although the catheter systems 100 illustrated herein are sometimes described as including a light source 124 and one or more light guides 122A, the catheter system 100 can alternatively include any suitable energy source and energy guides for purposes of generating the desired plasma in the balloon fluid 132 within the balloon interior 146 of each of the balloons 104. For example, in one non-exclusive alternative embodiment, the energy source 124 can be configured to provide high voltage pulses, and each energy guide 122A can include an electrode pair including spaced apart electrodes that extend into the balloon interior 146. In such embodiment, each pulse of high voltage is applied to the electrodes and forms an electrical arc across the electrodes, which, in turn, generates the plasma and forms the pressure waves within the balloon fluid 132 that are utilized to provide the fracture force onto the vascular lesions at the treatment site 106. Still alternatively, the energy source 124 and/or the energy guides 122A can have another suitable design and/or configuration.

The treatment system 142, such as via the outer shaft 110 and/or the inner shaft 111, can be coupled to the one or more energy guides 122A of the energy guide bundle 122 that are in optical communication with the energy source 124. The energy guide(s) 122A can be disposed along the inner tube 162 of each treatment device 143 and within the balloon 104. In some embodiments, each energy guide 122A can be an optical fiber and the energy source 124 can be a laser. The energy source 124 can be in optical communication with the energy guides 122A at the proximal portion 114 of the catheter system 100.

It is appreciated that the catheter system 100 and/or the energy guide bundle 122 can include any number of energy guides 122A in optical communication with the energy source 124 at the proximal portion 114, and with the balloon fluid 132 within the balloon interior 146 of each balloon 104 at the distal portion 116. For example, in some embodiments, the catheter system 100 and/or the energy guide bundle 122 can include from one energy guide 122A to five energy guides 122A that are usable within each treatment device 143. In other embodiments, the catheter system 100 and/or the energy guide bundle 122 can include from five energy guides 122A to fifteen energy guides 122A that are usable within each treatment device 143. In yet other embodiments, the catheter system 100 and/or the energy guide bundle 122 can include from ten energy guides 122A to thirty energy guides 122A that are usable within each treatment device 143. Alternatively, in still other embodiments, the catheter system 100 and/or the energy guide bundle 122 can include greater than thirty energy light guides 122A that are usable within each treatment device 143.

In some embodiments, the inner tube 162 of each treatment device 143 can be coupled to multiple energy guides 122A such as a first energy guide, a second energy guide, a third energy guide, etc., which can be disposed at any suitable positions about the inner tube 162 of each treatment device 143. For example, in certain non-exclusive embodiments, two energy guides 122A can be spaced apart by approximately 180 degrees about the circumference of the inner tube 162 of the respective treatment device 143; three energy guides 122A can be spaced apart by approximately 120 degrees about the circumference of the inner tube 162 of the respective treatment device 143; four energy guides 122A can be spaced apart by approximately 90 degrees about the circumference of the inner tube 162 of the respective treatment device 143; or six energy guides 122A can be spaced apart by approximately 60 degrees about the circumference of the inner tube 162 of the respective treatment device 143. Still alternatively, multiple energy guides 122A need not be uniformly spaced apart from one another about the circumference of the inner tube 162 of the respective treatment device 143. More particularly, it is further appreciated that the energy guides 122A can be disposed uniformly or non-uniformly about the inner tube 162 of the respective treatment device 143 to achieve the desired effect in the desired locations.

In some embodiments, the energy source 124 of the catheter system 100 can be configured to provide sub-millisecond pulses of energy from the energy source 124, along the energy guides 122A, to a location within the balloon interior 146 of each balloon 104, thereby inducing plasma formation in the balloon fluid 132 within the balloon interior 146 of each balloon 104, i.e. via a plasma generator 133 located at a guide distal end 122D of the energy guide 122A. The plasma formation causes rapid bubble formation, and imparts pressure waves upon the treatment site 106. Exemplary plasma-induced bubbles are shown as bubbles 134 in FIG. 1.

As noted above, the energy guides 122A can have any suitable design for purposes of generating plasma and/or pressure waves in the balloon fluid 132 within the balloon interior 146 of each balloon 104. Thus, the particular description of the light guides 122A herein is not intended to be limiting in any manner, except for as set forth in the claims appended hereto.

In certain embodiments, the energy guides 122A can include an optical fiber or flexible light pipe. The energy guides 122A can be thin and flexible and can allow light signals to be sent with very little loss of strength. The energy guides 122A can include a core surrounded by a cladding about its circumference. In some embodiments, the core can be a cylindrical core or a partially cylindrical core. The core and cladding of the energy guides 122A can be formed from one or more materials, including but not limited to one or more types of glass, silica, or one or more polymers. The energy guides 122A may also include a protective coating, such as a polymer. It is appreciated that the index of refraction of the core will be greater than the index of refraction of the cladding.

Each energy guide 122A can guide energy along its length from a proximal portion, i.e. a guide proximal end 122P, to a distal portion, i.e. the guide distal end 122D, having at least one optical window (not shown in FIG. 1) that is positioned within the balloon interior 146. The energy guides 122A can create an energy path as a portion of an optical network including the energy source 124. The energy path within the optical network allows energy to travel from one part of the network to another. Both the optical fiber and the flexible light pipe can provide an energy path within the optical networks herein.

The energy guides 122A can assume many configurations about and/or relative to the inner tube 162 of the treatment devices 143. In some embodiments, the energy guides 122A can run parallel to the longitudinal axis 144 of the inner shaft 111. In some embodiments, the energy guides 122A can be physically coupled to the inner tube 162 of the respective treatment device 143. In other embodiments, the energy guides 122A can be disposed along a length of an outer diameter of the inner tube 162 of the respective treatment device 143. In yet other embodiments, the energy guides 122A can be disposed within one or more energy guide lumens within or adjacent to the inner tube 162 of the respective treatment device 143.

It is further appreciated that the energy guides 122A can be disposed at any suitable positions about the circumference of the inner tube 162 of the respective treatment device 143, and the guide distal end 122D of each of the energy guides 122A can be disposed at any suitable longitudinal position relative to the length of the balloon 104 and/or relative to the length of the inner tube 162 of the respective treatment device 143.

In some embodiments, the energy guides 122A can include one or more photoacoustic transducers (not shown in FIG. 1), where each photoacoustic transducer can be in optical communication with the energy guide 122A within which it is disposed. In some embodiments, the photoacoustic transducers can be in optical communication with the guide distal end 122D of the energy guide 122A. In such embodiments, the photoacoustic transducers can have a shape that corresponds with and/or conforms to the guide distal end 122D of the energy guide 122A.

The photoacoustic transducer is configured to convert light energy into an acoustic wave at or near the guide distal end 122D of the energy guide 122A. It is appreciated that the direction of the acoustic wave can be tailored by changing an angle of the guide distal end 122D of the energy guide 122A.

It is further appreciated that the photoacoustic transducers disposed at the guide distal end 122D of the energy guide 122A can assume the same shape as the guide distal end 122D of the energy guide 122A. For example, in certain non-exclusive embodiments, the photoacoustic transducer and/or the guide distal end 122D can have a conical shape, a convex shape, a concave shape, a bulbous shape, a square shape, a stepped shape, a half-circle shape, an ovoid shape, and the like. It is also appreciated that the energy guide 122A can further include additional photoacoustic transducers disposed along one or more side surfaces of the length of the energy guide 122A.

The energy guides 122A can further include one or more diverting features or “diverters” (not shown in FIG. 1) within the energy guide 122A that are configured to direct light to exit the energy guide 122A toward a side surface, such as at or near the guide distal end 122D of the energy guide 122A, and toward the balloon wall 130. A diverting feature can include any feature of the system that diverts energy from the energy guide 122A away from its axial path toward a side surface of the energy guide 122A. The energy guides 122A can each include one or more energy windows disposed along the longitudinal or circumferential surfaces of each energy guide 122A and in optical communication with a diverting feature. Stated in another manner, the diverting features can be configured to direct energy in the energy guide 122A toward a side surface, such as at or near the guide distal end 122D, where the side surface is in optical communication with an energy window. The energy windows can include a portion of the energy guide 122A that allows energy to exit the energy guide 122A from within the energy guide 122A, such as a portion of the energy guide 122A lacking a cladding material on or about the energy guide 122A.

Examples of diverting features suitable for use herein include a reflecting element, a refracting element, and a fiber diffuser. Diverting features suitable for focusing light away from the tip of the energy guides 122A can include, but are not to be limited to, those having a convex surface, a gradient-index (GRIN) lens, and a mirror focus lens. Upon contact with the diverting feature, the light is diverted within the energy guide 122A to either a plasma generator 133 or the photoacoustic transducer that is in optical communication with a side surface of the energy guide 122A. As noted, the photoacoustic transducer then converts light energy into an acoustic wave that extends away from the side surface of the energy guide 122A.

The source manifold 136 can be positioned at or near the proximal portion 114 of the catheter system 100. The source manifold 136 can include one or more proximal end openings that can receive the plurality of energy guides 122A of the energy guide bundle 122, the guidewire 112, and/or an inflation conduit 140 that is coupled in fluid communication with the fluid pump 138. The catheter system 100 can also include the fluid pump 138 that is configured to inflate each balloon 104 with the balloon fluid 132, i.e. via the inflation conduit 140 and/or the inflation tubes 160, as needed.

As noted above, in the embodiment illustrated in FIG. 1, the system console 123 includes one or more of the energy source 124, the power source 125, the system controller 126, and the GUI 127. Alternatively, the system console 123 can include more components or fewer components than those specifically illustrated in FIG. 1. For example, in certain non-exclusive alternative embodiments, the system console 123 can be designed without the GUI 127. Still alternatively, one or more of the energy source 124, the power source 125, the system controller 126, and the GUI 127 can be provided within the catheter system 100 without the specific need for the system console 123.

As illustrated in FIG. 1, the system console 123 and the components included therewith are operatively coupled to the treatment system 142 and/or the catheter 102, the energy guide bundle 122, and the remainder of the catheter system 100. For example, in some embodiments, the system console 123 can include a console connection aperture 148 (also sometimes referred to generally as a “socket”) by which the energy guide bundle 122 is mechanically coupled to the system console 123. In such embodiments, the energy guide bundle 122 can include a guide coupling housing 150 (also sometimes referred to generally as a “ferrule”) that houses a portion, e.g., the guide proximal end 122P, of each of the energy guides 122A. The guide coupling housing 150 is configured to fit and be selectively retained within the console connection aperture 148 to provide the desired mechanical coupling between the energy guide bundle 122 and the system console 123.

The energy guide bundle 122 can also include a guide bundler 152 (or “shell”) that brings each of the individual energy guides 122A closer together so that the energy guides 122A and/or the energy guide bundle 122 can be in a more compact form as it extends with the treatment system 142 and/or the catheter 102 into the heart valve 108 during use of the catheter system 100.

The energy source 124 can be selectively and/or alternatively coupled in optical communication with each of the energy guides 122A, i.e. to the guide proximal end 122P of each of the energy guides 122A, in the energy guide bundle 122. In particular, the energy source 124 is configured to generate energy in the form of a source beam 124A, such as a pulsed source beam, that can be selectively and/or alternatively directed to and received by each of the energy guides 122A in the energy guide bundle 122 as an individual guide beam 124B. Alternatively, the catheter system 100 can include more than one energy source 124. For example, in one non-exclusive alternative embodiment, the catheter system 100 can include a separate energy source 124 for each of the energy guides 122A in the energy guide bundle 122.

The energy source 124 can have any suitable design. In certain embodiments, as noted above, the energy source 124 can be configured to provide sub-millisecond pulses of energy from the energy source 124 that are focused onto a small spot in order to couple it into the guide proximal end 122P of the energy guide 122A. Such pulses of energy are then directed along the energy guides 122A to a location within the balloon 104, thereby inducing plasma formation in the balloon fluid 132 within the balloon interior 146 of each balloon 104. In particular, the energy emitted at the guide distal end 122D of the energy guide 122A energizes the plasma generator 133 to form the plasma within the balloon fluid 132 within the balloon interior 146. The plasma formation causes rapid bubble formation, and imparts pressure waves upon the treatment site 106. In such embodiments, the sub-millisecond pulses of energy from the energy source 124 can be delivered to the treatment site 106 at a frequency of between approximately one hertz (Hz) and 5000 Hz. In some embodiments, the sub-millisecond pulses of energy from the energy source 124 can be delivered to the treatment site 106 at a frequency of between approximately 30 Hz and 1000 Hz. In other embodiments, the sub-millisecond pulses of energy from the energy source 124 can be delivered to the treatment site 106 at a frequency of between approximately ten Hz and 100 Hz. In yet other embodiments, the sub-millisecond pulses of energy from the energy source 124 can be delivered to the treatment site 106 at a frequency of between approximately one Hz and 30 Hz. Alternatively, the sub-millisecond pulses of energy can be delivered to the treatment site 106 at a frequency that can be greater than 5000 Hz.

It is appreciated that although the energy source 124 is typically utilized to provide pulses of energy, the energy source 124 can still be described as providing a single source beam 124A, i.e. a single pulsed source beam.

The energy sources 124 can include various types of light sources including lasers and lamps. Alternatively, as noted above, the energy sources 124, as referred to herein, can include any suitable type of energy source.

Certain suitable lasers can include short pulse lasers on the sub-millisecond timescale. In some embodiments, the energy source 124 can include lasers on the nanosecond (ns) timescale. The lasers can also include short pulse lasers on the picosecond (ps), femtosecond (fs), and microsecond (us) timescales. It is appreciated that there are many combinations of laser wavelengths, pulse widths and energy levels that can be employed to achieve plasma in the balloon fluid 132 of the treatment systems 142. In various embodiments, the pulse widths can include those falling within a range including from at least ten ns to 3000 ns. In some embodiments, the pulse widths can include those falling within a range including from at least 20 ns to 100 ns. In other embodiments, the pulse widths can include those falling within a range including from at least one ns to 500 ns.

Exemplary nanosecond lasers can include those within the UV to IR spectrum, spanning wavelengths of about ten nanometers (nm) to one millimeter (mm). In some embodiments, the energy sources 124 suitable for use in the catheter systems 100 can include those capable of producing light at wavelengths of from at least 750 nm to 2000 nm. In other embodiments, the energy sources 124 can include those capable of producing light at wavelengths of from at least 700 nm to 3000 nm. In still other embodiments, the energy sources 124 can include those capable of producing light at wavelengths of from at least 100 nm to ten micrometers (μm). Nanosecond lasers can include those having repetition rates of up to 200 kHz. In some embodiments, the laser can include a Q-switched thulium:yttrium-aluminum-garnet (Tm:YAG) laser. In other embodiments, the laser can include a neodymium:yttrium-aluminum-garnet (Nd:YAG) laser, holmium:yttrium-aluminum-garnet (Ho:YAG) laser, erbium:yttrium-aluminum-garnet (Er:YAG) laser, excimer laser, helium-neon laser, carbon dioxide laser, as well as doped, pulsed, fiber lasers.

The catheter systems 100 can generate pressure waves having maximum pressures in the range of at least one megapascal (MPa) to 100 MPa. The maximum pressure generated by a particular catheter system 100 will depend on the energy source 124, the absorbing material, the bubble expansion, the propagation medium, the balloon material, and other factors. In some embodiments, the catheter systems 100 can generate pressure waves having maximum pressures in the range of at least two MPa to 50 MPa. In other embodiments, the catheter systems 100 can generate pressure waves having maximum pressures in the range of at least two MPa to 30 MPa. In yet other embodiments, the catheter systems 100 can generate pressure waves having maximum pressures in the range of at least 15 MPa to 25 MPa.

The pressure waves can be imparted upon the treatment site 106 from a distance within a range from at least 0.1 millimeters (mm) to 25 mm extending radially from the energy guides 122A when the treatment devices 143 are placed at the treatment site 106. In some embodiments, the pressure waves can be imparted upon the treatment site 106 from a distance within a range from at least ten mm to 20 mm extending radially from the energy guides 122A when the treatment devices 143 are placed at the treatment site 106. In various embodiments, the pressure waves can be imparted upon the treatment site 106 from a distance within a range from at least one mm to ten mm extending radially from the energy guides 122A when the treatment devices 143 are placed at the treatment site 106. In certain embodiments, the pressure waves can be imparted upon the treatment site 106 from a distance within a range from at least 1.5 mm to four mm extending radially from the energy guides 122A when the treatment devices 143 are placed at the treatment site 106. In some embodiments, the pressure waves can be imparted upon the treatment site 106 from a range of at least two MPa to 30 MPa at a distance from 0.1 mm to ten mm. In some embodiments, the pressure waves can be imparted upon the treatment site 106 from a range of at least two MPa to 25 MPa at a distance from 0.1 mm to ten mm.

The power source 125 is electrically coupled to and is configured to provide necessary power to each of the energy source 124, the system controller 126, the GUI 127, the handle assembly 128, and the treatment system 142. The power source 125 can have any suitable design for such purposes.

As noted, the system controller 126 is electrically coupled to and receives power from the power source 125. The system controller 126 is coupled to and is configured to control operation of each of the energy source 124, the GUI 127 and the treatment system 142. The system controller 126 can include one or more processors or circuits for purposes of controlling the operation of at least the energy source 124, the GUI 127 and the treatment system 142. For example, the system controller 126 can control the energy source 124 for generating pulses of energy as desired, e.g., at any desired firing rate. The system controller 126 can control and/or operate in conjunction with the treatment system 142 to effectively and efficiently provide the desired fracture forces adjacent to and/or on or between adjacent leaflets 1088 within the heart valve 108 at the treatment site 106.

The system controller 126 can further be configured to control operation of other components of the catheter system 100, such as the positioning of the treatment system 142 and/or the catheter 102 adjacent to the treatment site 106, the inflation of each balloon 104 with the balloon fluid 132, etc. The catheter system 100 can include one or more additional controllers that can be positioned in any suitable manner for purposes of controlling the various operations of the catheter system 100. For example, in certain embodiments, an additional controller and/or a portion of the system controller 126 can be positioned and/or incorporated within the handle assembly 128.

The GUI 127 is accessible by the user or operator of the catheter system 100. The GUI 127 is can be electrically connected to the system controller 126. With this design, the GUI 127 can be used by the user or operator to ensure that the catheter system 100 is employed as desired to impart pressure onto and induce fractures into the vascular lesions at the treatment site 106. The GUI 127 can provide the user or operator with information that can be used before, during and after use of the catheter system 100. In one embodiment, the GUI 127 can provide static visual data and/or information to the user or operator. In addition, or in the alternative, the GUI 127 can provide dynamic visual data and/or information to the user or operator, such as video data or any other data that changes over time, such as during use of the catheter system 100. In various embodiments, the GUI 127 can include one or more colors, different sizes, varying brightness, etc., that may act as alerts to the user or operator. The GUI 127 can provide audio data or information to the user or operator. It is appreciated that the specifics of the GUI 127 can vary depending upon the design requirements of the catheter system 100, or the specific needs, specifications and/or desires of the user or operator.

As shown in FIG. 1, the handle assembly 128 can be positioned at or near the proximal portion 114 of the catheter system 100, and/or near the source manifold 136. In this embodiment, the handle assembly 128 is coupled to each balloon 104 and is positioned spaced apart from each balloon 104. Alternatively, the handle assembly 128 can be positioned at another suitable location.

The handle assembly 128 is handled and used by the user or operator to operate, position and control the treatment system 142 and/or the catheter 102. The design and specific features of the handle assembly 128 can vary to suit the design requirements of the catheter system 100. In the embodiment illustrated in FIG. 1, the handle assembly 128 is separate from, but in electrical and/or fluid communication with one or more of the system controller 126, the energy source 124, the fluid pump 138, the GUI 127 and the treatment system 142. In some embodiments, the handle assembly 128 can integrate and/or include at least a portion of the system controller 126 within an interior of the handle assembly 128. For example, in certain such embodiments, the handle assembly 128 can include circuitry 156 that can form at least a portion of the system controller 126. In one embodiment, the circuitry 156 can include a printed circuit board having one or more integrated circuits, or any other suitable circuitry. In an alternative embodiment, the circuitry 156 can be omitted, or can be included within the system controller 126, which in various embodiments can be positioned outside of the handle assembly 128, e.g., within the system console 123. It is understood that the handle assembly 128 can include fewer or additional components than those specifically illustrated and described herein.

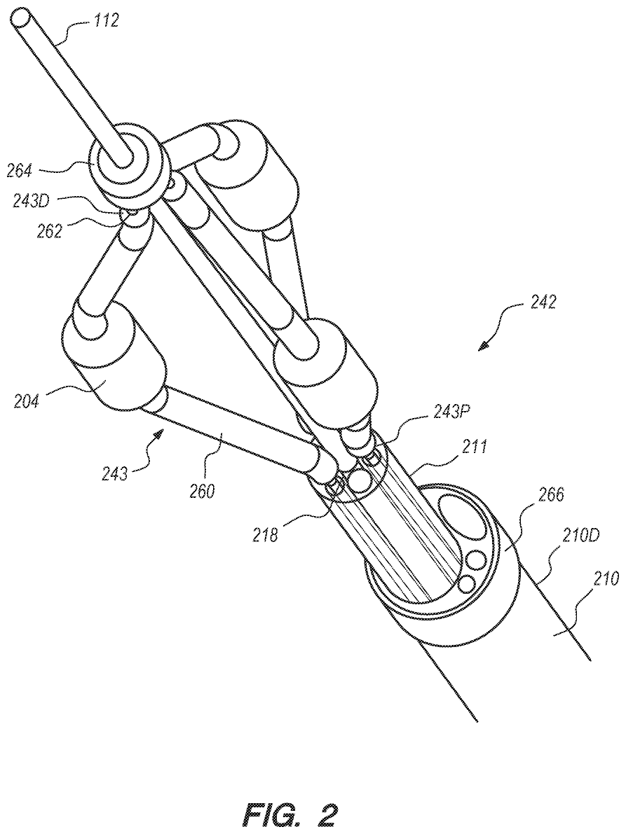

Descriptions of various embodiments and implementations of the treatment system 142, and usages thereof, are described in detail herein below, such as shown in FIGS. 2-10. However, it is further appreciated that alternative embodiments and implementations may also be employed that would be apparent to those skilled in the relevant art based on the teachings provided herein. Thus, the scope of the present embodiments and implementations is not intended to be limited to just those specifically described herein, except as recited in the claims appended hereto.

FIG. 2 is a simplified perspective view of a portion of an embodiment of the valvuloplasty treatment system 242. As illustrated in FIG. 2, in various embodiments, the treatment system 242 includes five basic components: the multi-lumen outer shaft 210, the external cap 266, the movable multi-lumen inner shaft 211, the deployment collet 264, and the plurality of spaced apart, individual treatment devices 243. Alternatively, the treatment system 242 can include more components or fewer components than those specifically illustrated and described herein. For example, in one non-exclusive alternative embodiment, as noted above, the treatment system 242 can be designed without the external cap 266. FIG. 2 also illustrates the guidewire 112 that extends through a guidewire lumen 218 formed into the inner shaft 211, with the deployment collet 264 being fixedly secured to the guidewire 112.

As provided above, the treatment system 242 is configured to impart pressure waves and/or fracture forces within each of the individual treatment devices 243 adjacent to the valve wall 108A (illustrated in FIG. 1) and/or on or between adjacent leaflets 1088 (illustrated in FIG. 1) within the heart valve 108 (illustrated in FIG. 1) at the treatment site 106 (illustrated in FIG. 1). Such pressure waves and/or fracture forces are utilized to break apart the vascular lesions that are located at the treatment site 106. It is also appreciated that the design of each of the components of the treatment system 242 can be varied to suit the requirements of the catheter system with which the treatment system 242 is being used.

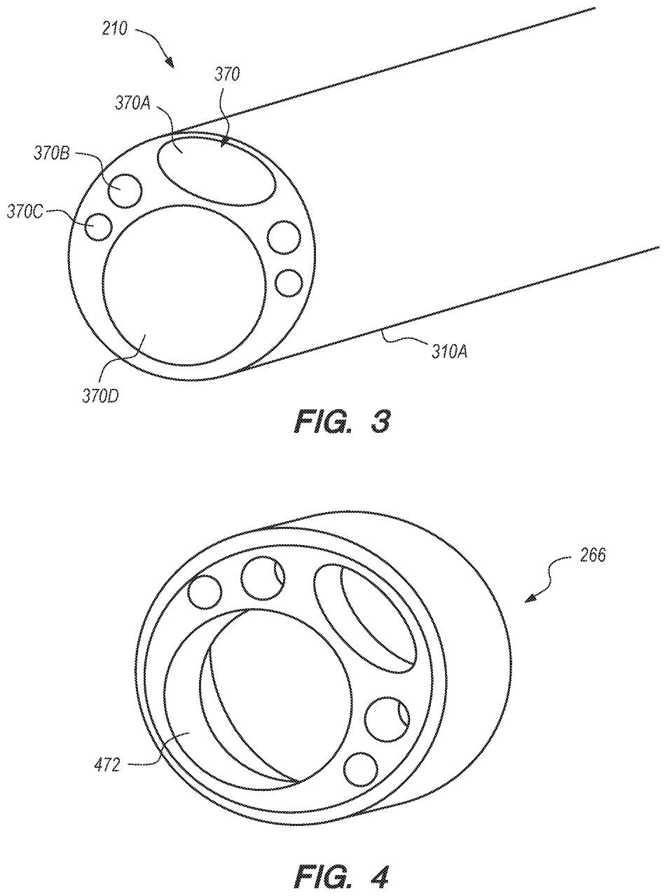

During deployment of the treatment system 242, the outer shaft 210 can be initially inserted into the body 107 (illustrated in FIG. 1) of the patient 109 (illustrated in FIG. 1), such as via an artery or other suitable blood vessel, so that the outer shaft 210 is positioned a predetermined distance, such as 10-15 millimeters or another suitable distance, away from the heart valve 108, i.e. away from the treatment site 106 within or adjacent to the heart valve 108. Referring now to FIG. 3, FIG. 3 is a simplified perspective view of a portion of the multi-lumen outer shaft 210 that can form part of the valvuloplasty treatment system 242 illustrated in FIG. 2.

As noted, the design of the outer shaft 210 can be varied to suit the specific requirements of the catheter system 100 (illustrated in FIG. 1). As illustrated in FIG. 3, the outer shaft 210 includes an outer shaft body 310A that defines a plurality of outer shaft lumens 370.

The outer shaft body 310A can have any suitable design and can be made from any suitable materials. For example, in various implementations, the outer shaft body 310A can be an articulated and braided shaft or tubing that is substantially cylindrical-shaped and can be formed from a flexible polymer material. Alternatively, the outer shaft body 310A can have another suitable design and/or can be formed from other suitable materials.

The plurality of outer shaft lumens 370 can be utilized for various purposes to enhance the operation of the treatment system 242. In the embodiment illustrated in FIG. 3, the outer shaft body 310A defines one or more first outer shaft lumens 370A, one or more second outer shaft lumens 370B, one or more third outer shaft lumens 370C, and a fourth outer shaft lumen 370D (also sometimes referred to as a “working channel”). Each of the outer shaft lumens 370A, 370B, 370C, 370D can be specifically configured to be used for different purposes to enhance the operation of the treatment system 242.

In one embodiment, as illustrated in FIG. 3, the outer shaft 210 can be designed with only a single first outer shaft lumen 370A. Alternatively, the outer shaft 210 can be designed to include more than one first outer shaft lumen 370A. In certain embodiments, the first outer shaft lumen 370A can be an imaging channel that is configured to enable real-time imaging of the treatment site 106 (illustrated in FIG. 1) while the treatment therapy is applied. More particularly, in one such embodiment, the first outer shaft lumen 370A can be an imaging channel that is configured to provide a complementary metal oxide semiconductor (CMOS) sensor housing with integrated LED or fiber optic lighting or an ultrasound chip to provide real-time imaging while the treatment therapy is applied. Alternatively, the first outer shaft lumen 370A can provide an imaging channel for a different type of imaging system.

In one non-exclusive embodiment, the one or more second outer shaft lumens 370B can be configured to function as irrigation ports usable for providing a cleaning solution, such as a saline solution, to clean a lens of the CMOS imaging system. Alternatively, the second outer shaft lumens 370B can be configured for another suitable purpose.

In one non-exclusive embodiment, the one or more third outer shaft lumens 370C can be configured as articulating lumens through which articulating wires can be employed for steering the outer shaft 210 as desired during placement and positioning of the outer shaft 210 relative to the treatment site 106.

The fourth outer shaft lumen 370D, i.e. the working channel, is configured to provide a channel through which the inner shaft 211 (illustrated in FIG. 2) is movably positioned relative to the treatment site 106. It is appreciated that the fourth outer shaft lumen 370D is sized and shaped to receive the inner shaft 211, while still allowing the inner shaft 211 to move through the fourth outer shaft lumen 370D for properly positioning the inner shaft 211 as desired.

It is further appreciated that the use and designation of the “first outer shaft lumens”, the “second outer shaft lumens”, the “third outer shaft lumens”, and the “fourth outer shaft lumen” is merely for convenience and ease of illustration, and any of the outer shaft lumens 370 can be referred to as “first outer shaft lumens”, “second outer shaft lumens”, “third outer shaft lumens”, and/or “fourth outer shaft lumens”.

Referring back now to FIG. 2, in certain embodiments, the treatment system 242 can include the external cap 266 that is configured to fit over an outer shaft distal end 210D of the outer shaft 210 to further enhance and/or stabilize relative movement between the inner shaft 211 and the outer shaft 210. More particularly, in certain embodiments, the external cap 266 is mounted at the outer shaft distal end 210D to which the articulating wires can be welded or otherwise attached.

FIG. 4 is a simplified perspective view of the external cap 266 that can form part of the valvuloplasty treatment system 242 illustrated in FIG. 2. The design of the external cap 266 can be varied to suit the requirements of the outer shaft 210 (illustrated in FIG. 2) and/or the catheter system 100 (illustrated in FIG. 1). As illustrated in FIG. 4, the external cap 266 can be configured to include a plurality of external cap apertures 472 that are specifically designed to coincide and/or align with the various outer shaft lumens 370 (illustrated in FIG. 3). More particularly, as shown, the external cap 266 includes external cap apertures 472 having a size and shape that is substantially similar to the size and shape of each of the first outer shaft lumen 370A (illustrated in FIG. 3), the second outer shaft lumens 370B (illustrated in FIG. 3), the third outer shaft lumens 370C (illustrated in FIG. 3), and the fourth outer shaft lumen 370D (illustrated in FIG. 3).

The external cap 266 can be made from any suitable materials. For example, in certain non-exclusive embodiments, the external cap 266 can be formed from plastic, metal or other suitable materials.

Referring again to FIG. 2, the inner shaft 211 is movably positioned within the outer shaft 210. In particular, during deployment of the treatment system 242, after the outer shaft 210 has been positioned as noted above, the inner shaft 211, with the guidewire 112, is inserted through the working channel 370D (illustrated in FIG. 3) of the outer shaft 210 and advanced past the leaflets 108B (illustrated in FIG. 1) of the heart valve 108 (illustrated in FIG. 1) and into the right heart atrium of the heart. More specifically, in certain applications, the inner shaft 211 can be inserted such that the treatment devices 243 are positioned so that the leaflets 1088 of the heart valve 108 are close to a middle of the balloon 204 of each treatment device 243.

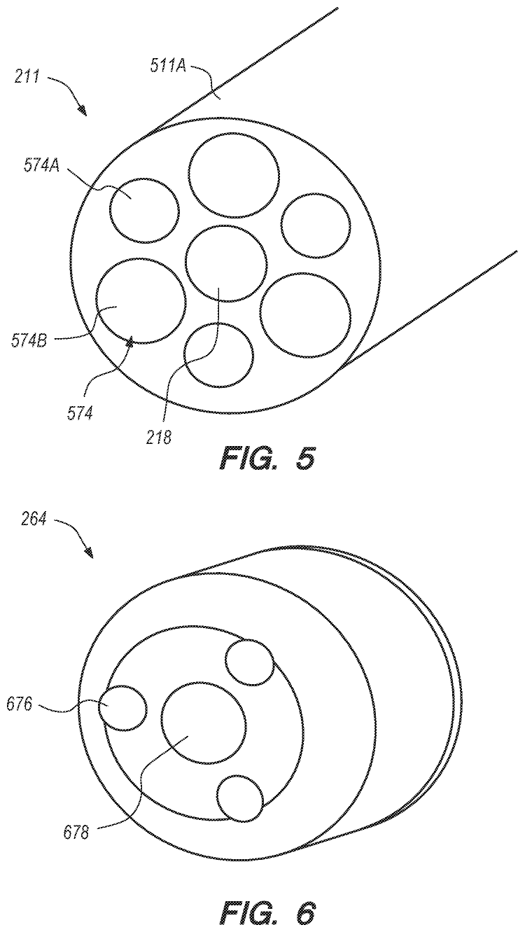

FIG. 5 is a simplified perspective view of a portion of the movable multi-lumen inner shaft 211 that can form part of the valvuloplasty treatment system 242 illustrated in FIG. 2. As noted, the design of the inner shaft 211 can be varied to suit the specific requirements of the catheter system 100 (illustrated in FIG. 1). As illustrated in FIG. 5, the inner shaft 211 includes an inner shaft body 511A that defines a plurality of inner shaft lumens 574.

The inner shaft body 511A can have any suitable design and can be made from any suitable materials. For example, in various implementations, the inner shaft body 511A can be a braided shaft or tubing that is substantially cylindrical-shaped and can be formed from a flexible polymer material. Alternatively, the inner shaft body 511A can have another suitable design and/or can be formed from other suitable materials.

The plurality of inner shaft lumens 574 can be utilized for various purposes to enhance the operation of the treatment system 242. In the embodiment illustrated in FIG. 5, the inner shaft body 511A defines a plurality of first inner shaft lumens 574A, a plurality of second inner shaft lumens 574B, and the guidewire lumen 218. Each of the inner shaft lumens 574A, 574B, 218 can be specifically configured to be used for different purposes to enhance the operation of the treatment system 242.

In certain embodiments, the plurality of first inner shaft lumens 574A can be configured for purposes substantially similar to one or more of the first outer shaft lumens 370A (illustrated in FIG. 3), the second outer shaft lumens 370B (illustrated in FIG. 3), and/or the third outer shaft lumens 370C (illustrated in FIG. 3). More particularly, in alternative implementations, the plurality of first inner shaft lumens 574A can function as (i) imaging channels that are configured to enable real-time imaging of the treatment site 106 (illustrated in FIG. 1) while the treatment therapy is applied; (ii) irrigation ports usable for providing a cleaning solution to clean a lens of the imaging system; and/or (iii) articulating lumens through which articulating wires can be employed for steering the inner shaft 211 as desired during placement and positioning of the inner shaft 211 relative to the treatment site 106. Alternatively, the first inner shaft lumens 574A can be used for other suitable purposes.

The plurality of second inner shaft lumens 574B can be configured as inflation ports that are used to inflate the balloons 204 (illustrated in FIG. 2) of each of the treatment devices 243 (illustrated in FIG. 2). More specifically, in the embodiment illustrated in FIG. 5, the inner shaft body 511A defines three second inner shaft lumens 574B, with one second inner shaft lumen 574B being utilized as an inflation port for each of the three treatment devices 243, i.e. with one treatment device 243 being operatively coupled to each of the three second inner shaft lumens 574B.

The guidewire lumen 218 provides a channel through which the guidewire 112 extends in order to guide placement of the treatment system 242 (illustrated in FIG. 2), the inner shaft 211, and/or the individual treatment devices 243 relative to the treatment site 106.

It is appreciated that the use and designation of the “first inner shaft lumens”, and the “second outer shaft lumens” is merely for convenience and ease of illustration, and any of the inner shaft lumens 574 can be referred to as “first outer shaft lumens”, and/or “second outer shaft lumens”.

Referring again to FIG. 2, the inner tube 262 of each treatment device 243 can be coupled to the deployment collet 264 at a device distal end 243D of the treatment device 243. The deployment collet 264 can be fixedly coupled to the guidewire 112.

FIG. 6 is a simplified perspective view of the deployment collet 264 that can form part of the valvuloplasty treatment system 242 illustrated in FIG. 2. The design of the deployment collet 264 can be varied. As illustrated in FIG. 6, the deployment collet 264 can include a plurality of device apertures 676, and a guidewire aperture 678.

In this embodiment, each of the device apertures 676 is configured to receive and retain a portion of the inner tube 262 (illustrated in FIG. 2) of one of the treatment devices 243 (illustrated in FIG. 2). Thus, with such design, the device distal end 243D (illustrated in FIG. 2) of each of the treatment devices 243 can be securely coupled to the deployment collet 264. With this design, movement of the guidewire 112 relative to the inner shaft 211 (illustrated in FIG. 2) during positioning and deployment of the treatment system 242 (illustrated in FIG. 2) results in the outwardly movement of the treatment devices 243 such that the treatment devices 243 can be effectively positioned adjacent to the leaflets 1088 (illustrated in FIG. 1) of the heart valve 108 (illustrated in FIG. 1) at the treatment site 106 (illustrated in FIG. 1).

In one embodiment, i.e. when the treatment devices 243 are equally spaced apart from one another, the device apertures 676 can be spaced apart from one another by approximately 120 degrees about the deployment collet 264. Alternatively, the device apertures 676 can be positioned relative to one another in another suitable manner depending on the desired positioning of the treatment devices 243.

The guidewire aperture 678 is sized and shaped so that the guidewire 112 can be extended through the guidewire aperture 678. The guidewire aperture 678 can be further configured so that the deployment collet 264 is fixedly secured to the guidewire 112, such that movement of the guidewire 112 results in corresponding movement of the deployment collet 264.

The deployment collet 264 can be made from any suitable materials. For example, in certain non-exclusive embodiments, the deployment collet 264 can be formed from plastic, metal or other suitable materials.

Referring again to FIG. 2, the treatment system 242 incudes the plurality of treatment devices 243, such as three spaced apart, individual treatment devices 243 in this particular embodiment, which are configured to impart pressure waves and/or fracture forces at specific locations adjacent to the valve wall 108A and/or on or between adjacent leaflets 108B within the heart valve 108 at the treatment site 106 in order to break apart the vascular lesions that are located at the treatment site 106. In one embodiment, as shown, each of the three treatment devices 243 can be positioned and/or mounted so as to be spaced apart by approximately 120 degrees from one another about and/or relative to the guidewire 112. Alternatively, the treatment devices 243 can be spaced apart from one another in a different manner.

The treatment devices 243 can be coupled at opposite ends to the inner shaft 211 and the deployment collet 264. More specifically, as shown in FIG. 2, each treatment device 243 can include an inflation tube 260 that is movably coupled to the inner shaft 211 at or near the device proximal end 243P, and an inner tube 262 that is coupled to the deployment collet 264 at or near the device distal end 243D.

Each treatment device 243 can further include a balloon 204 that is coupled to the inflation tube 260 and/or the inner tube 262.

Each of the treatment devices 243 can also include one or more energy guides 722A (illustrated, for example, in FIG. 7B) that are positioned and utilized to generate the desired pressure waves and/or fracture forces in the balloon fluid 132 (illustrated in FIG. 1) within the balloon interior 746 (illustrated, for example, in FIG. 7B) of each balloon 204.

It is appreciated that the treatment devices 243, and thus the balloons 204, once deployed, can be rotated as necessary such that the treatment devices 243 are properly lined up so that the desired pressure waves and/or fracture forces can be more precisely directed and focused between the leaflets 108B of the heart valve 108. It is further appreciated that the desired pressure waves and/or fracture forces can be deployed from a few millimeters diameter to over 35 millimeters depending upon the size of the heart valve 108.

FIG. 7A is a simplified perspective view of a portion of the multi-lumen outer shaft 210, a portion of the movable multi-lumen inner shaft 211, and a portion of one treatment device 243 that can form a part of the valvuloplasty treatment system 242 illustrated in FIG. 2. It is appreciated that although only one treatment device 243 is shown in FIG. 7A, the treatment system 242 will typically include a plurality of treatment devices 243, e.g., three treatment devices 243.

As illustrated in FIG. 7A, the treatment device 243 is shown in a first (retracted) position. More particularly, the treatment device 243, including the balloon 204, is coupled into one of the second inner shaft lumens 574B that are formed into the inner shaft body 511A of the inner shaft 211, such as with a device coupler 757. In certain embodiments, the device coupler 757 can be provided in the form of a flared-out collar, with a narrower first coupler end 757A that extends into the second inner shaft lumen 574B, and an opposed flared (and thus wider) second coupler end 757B to which the treatment device 243 and/or the balloon 204 is coupled. Alternatively, the device coupler 757 can have a different design for purposes of effectively coupling the treatment device 243 to the inner shaft 211.

The device coupler 757 can be formed from any suitable materials. For example, in some non-exclusive embodiments, the device coupler 757 can be formed from one of a metal material or a polymer material. Alternatively, the device coupler 757 can be formed from other suitable materials.

As shown in FIG. 7A, during insertion of the inner shaft 211 through the working channel 370D formed into the outer shaft body 310A of the outer shaft 210, the balloon 204 of the treatment device 243 is pulled back so as to be anchored onto the device coupler 757. With such positioning of the treatment device 243 relative to the inner shaft 211, the inner shaft 211 and the treatment device 243 can be more easily moved as desired into a desired position adjacent to the treatment site 106 (illustrated in FIG. 1) within the body 107 (illustrated in FIG. 1) of the patient 109.

FIG. 7B is another simplified perspective view of a portion of the multi-lumen outer shaft 210, the movable multi-lumen inner shaft 211, and the treatment device 243 illustrated in FIG. 7A that can form a part of the valvuloplasty treatment system 242. However, in FIG. 7B, the treatment device 243 is now shown in a second (extended) position. In particular, as illustrated, the treatment device 243 and/or the balloon 204 has now been pushed out from the second inner shaft lumen 574B that is formed into the inner shaft body 511A of the inner shaft 211. More specifically, the inflation tube 260 of the treatment device 243 is shown as being coupled to the inner shaft 211, i.e. with the inflation tube 260 extending into and/or through the device coupler 757. In this embodiment, the balloon proximal end 704P of the balloon 204 is shown coupled to the inflation tube 260.

It is appreciated that the balloon 204 is illustrated in a translucent manner in FIG. 7B so that additional components of the treatment device 243 can be more clearly illustrated and described. More particularly, as shown in FIG. 7B, the treatment device 243 further includes the inflation tube 260, the inner tube 262, a guide positioner 780, a portion of one or more of the energy guides 722A, and one or more plasma target rings 782. Alternatively, the treatment device 243 can include more components or fewer components than what is specifically shown in FIG. 7B. For example, in certain alternative embodiments, the treatment device 243 can be designed without the guide positioner 780 and/or the plasma target rings 782.

The inflation tube 260 is movably coupled to the inner shaft 211, such as via the device coupler 757, at or near the device proximal end 243P. The inflation tube 260 can be used as a conduit through which the balloon fluid 132 (illustrated in FIG. 1) can be transmitted into the balloon interior 746 of the balloon 204 in order to expand the balloon 204 from the deflated configuration to the inflated configuration.

The inflation tube 260 can have any suitable design and can be made from any suitable materials. For example, in various implementations, the inflation tube 260 can be a substantially cylindrical-shaped tube that can be formed from a flexible polymer material. Alternatively, the inflation tube 260 can have another suitable design and/or can be formed from other suitable materials.

In certain embodiments, the inner tube 262 can be configured to extend substantially the entire length of the treatment device 243, with the inner tube 262 being coupled to the deployment collet 264 (illustrated in FIG. 2) at or near the device distal end 243D.

The inner tube 262 can have any suitable design and can be made from any suitable materials. For example, in various implementations, the inner tube 262 can be a substantially cylindrical-shaped tube that can be formed from a flexible polymer material. Alternatively, the inner tube 262 can have another suitable design and/or can be formed from other suitable materials.

As shown in FIG. 7B, the guide positioner 780 is positioned substantially about the inner tube 262. In one embodiment, the guide positioner 780 is configured to define a plurality of grooves about the inner tube 262 to provide specific positioning control for each of the one or more energy guides 722A that may be used within the treatment device 243. The guide positioner 780 can be configured to define any suitable number of grooves for providing specific positioning control of any suitable number of energy guides 722A. For example, in one embodiment, the guide positioner 780 can be configured to define six grooves for providing specific positioning control of up to six energy guides 722A. Alternatively, the guide positioner 780 can be configured to define greater than six or fewer than six grooves for providing specific positioning control of up to greater than six or fewer than six energy guides 722A.

The guide positioner 780 can be made from any suitable materials. For example, in various implementations, the guide positioner 780 can be formed from a flexible polymer material. Alternatively, the guide positioner 780 can be formed from other suitable materials.

The treatment device 243 can include one or more energy guides 722A that are configured to guide energy from the energy source 124 (illustrated in FIG. 1) to induce plasma formation in the balloon fluid 132 within the balloon interior 746 of the balloon 204, i.e. via a plasma generator such as the plasma target rings 782 located at or near a guide distal end 722D of the energy guide 722A. The plasma formation causes rapid bubble formation, and imparts pressure waves upon the treatment site 106 (illustrated in FIG. 1).

In certain embodiments, the plasma target rings 782 can be used to generate the desired plasma in the balloon fluid 132 within the balloon interior 746.

FIG. 7C is still another simplified perspective view of a portion of the treatment device 243 illustrated in FIG. 7A. In particular, FIG. 7C provides a different perspective view, and thus additional details, of the balloon 204 (again illustrated as transparent for clarity), the inflation tube 260, the inner tube 262, the guide positioner 780, the one or more of the energy guides 722A, and the one or more plasma target rings 782 of the treatment device 243.

FIG. 7D is yet another simplified perspective view of a portion of the treatment device illustrated in FIG. 7A. In particular, FIG. 7D provides an enlarged perspective view, and thus additional details, of the inner tube 262, the guide positioner 780, the one or more of the energy guides 722A, and the one or more plasma target rings 782 of the treatment device 243.

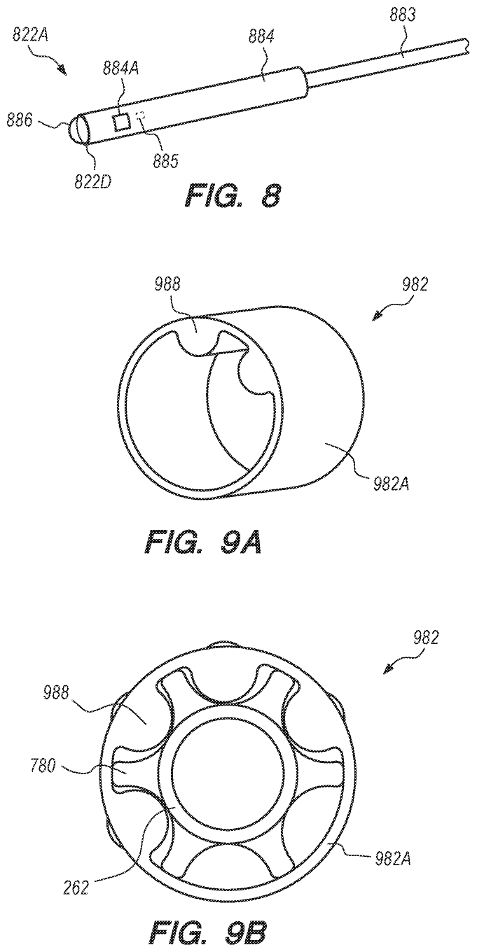

FIG. 8 is a simplified perspective view of a portion of an energy guide 822A usable as part of the treatment device 243 illustrated in FIG. 7A. As noted above, the energy guide 822A can have any suitable design for purposes of guiding energy from the energy source 124 (illustrated in FIG. 1) into the balloon interior 746 (illustrated in FIG. 7B) of each balloon 204 (illustrated in FIG. 2) to induce plasma generation, and thus desired pressure waves, in the balloon fluid 132 (illustrated in FIG. 1) within the balloon interior 746 of each balloon 204.

In some embodiments, the energy guides 822A can include an optical fiber or flexible light pipe, which is thin and flexible and is configured to allow energy to be sent through the energy guide 822A with very little loss of strength. The energy guide 822A can include a guide core 883 that is surrounded, at least in part, by a guide housing 884. In one embodiment, the guide core 883 can be a cylindrical core or a partially cylindrical core. The energy guide 822A may also include a protective coating, such as a polymer.

As shown, in certain embodiments, the energy guide 822A and/or the guide housing 884 can include at least one optical window 884A positioned near the guide distal end 822D of the energy guide 822A. The optical window 884A can include a portion of the energy guide 822A and/or the guide housing 884 that allows energy to exit the guide housing 884 from within the guide housing 844, such as a portion of the guide housing 884 lacking a cladding material on or about the guide housing 884.

In some embodiments, the energy guide 822A can include one or more photoacoustic transducers 885 (illustrated in phantom), where each photoacoustic transducer 885 can be in optical communication with the energy guide 822A within which it is disposed. The photoacoustic transducer 885 is configured to convert light energy into an acoustic wave at or near the guide distal end 822D of the energy guide 822A.

In certain embodiments, as noted above, the energy guide 822A can include one or more diverters (not shown) within the guide housing 844 that are configured to direct energy to exit the guide housing 884 toward a side surface, such as through the optical window 884A.

In some embodiments, the energy guide 822A can also include an optical element 886 that is positioned at or near the guide distal end 822D of the energy guide 822A. With such design, instead of the energy being directed outwardly through the optical window 884A, the energy being transmitted through the energy guide 822A can exit the energy guide 822A through the optical element 886 such that the energy is directed toward one of the plasma target rings 782 (illustrated in FIG. 7B). The energy from the energy guide 822A impinging on a plasma target 988 (illustrated in FIG. 9A) of the plasma target ring 782 generates the desired plasma in the balloon fluid 132 within the balloon interior 746 of the balloon 204.

In one embodiment, the optical element 886 can include an optically clear lens that is configured to protect the guide distal end 822D of the energy guide 822A. Alternatively, the optical element 886 can have another suitable design.

FIG. 9A is a simplified perspective view of an embodiment of a plasma target ring 982 usable as part of the treatment device 243 illustrated in FIG. 7A. FIG. 9B is a simplified end view of another embodiment of the plasma target ring 982 illustrated in FIG. 9A, and a portion of the inner tube 262 and the guide positioner 780 that are usable as part of the treatment device 243.

The design of the plasma target ring 982 can be varied to suit the requirements of the treatment device 243. In certain embodiments, the plasma target ring 982 can have a ring-shaped ring body 982A that is configured to slide over the inner tube 262 and the guide positioner 780. The plasma target ring 982 can include one or more plasma targets 988 that are configured to convert energy directed from the energy guide 822A (illustrated in FIG. 8), e.g., directed through the optical element 886 (illustrated in FIG. 8), to an energy wave, such as an ultrasonic soundwave, in order to break apart the calcified lesions at the treatment site 106 (illustrated in FIG. 1). In one embodiment, the plasma target ring 982 can be formed from a machined metal rod that is slid over the grooved inner tube 262 and/or guide positioner 780. The plasma target ring 982 can then be swaged (appropriately shaped) or glued down onto the inner tube 262 and/or guide positioner 780. Alternatively, the plasma target ring 982 can have another suitable design and/or can be positioned in another suitable manner.

The plasma target ring 982 and/or the plasma targets 988 can be formed from various materials. In some embodiments, the plasma target ring 982 and/or the plasma targets 988 can be formed from metallics and/or metal alloys having relatively high melting temperatures, such as tungsten, tantalum, molybdenum, niobium, platinum and/or iridium. Alternatively, the plasma target ring 982 and/or the plasma targets 988 can be formed from at least one of magnesium oxide, beryllium oxide, tungsten carbide, titanium nitride, titanium carbonitride and titanium carbide. Still alternatively, the plasma target ring 982 and/or the plasma targets 988 can be formed from at least one of diamond CVD and diamond. In other embodiments, the plasma target ring 982 and/or the plasma targets 988 can be formed from transition metal, an alloy metal or a ceramic material. Still alternatively, the plasma target ring 982 and/or the plasma targets 988 can be formed from any other suitable material(s).

As illustrated in FIG. 7B, the plasma target ring 982 is positioned such that the plasma target ring 982, and thus the plasma targets 988, is spaced apart from the guide distal end 722D of the energy guides 722A. In certain embodiments, the respective plasma target 988 can be spaced apart from the guide distal end 722D of the energy guide 722A by a target gap distance of at least between 1 μm and 1 cm. For example, in some non-exclusive such embodiments, the target gap distance can be at least 1 μm, at least 10 μm, at least 100 μm, at least 1 mm, at least 2 mm, at least 3 mm, at least 5 mm or at least 1 cm. The target gap distance can vary depending upon the size, shape and/or angle of the plasma target 988 relative to the energy emitted by the energy guide 722A, the type of material used to form the plasma target 988, the quantity and/or duration of the energy being emitted from the energy guide 722A, the type of balloon fluid 132 (illustrated in FIG. 1) used in the balloon 204 (illustrated in FIG. 2), etc.

During use of the treatment device 243, the energy directed from the energy guide 722A impinges on the plasma target 988 to generate a plasma bubble 134 (illustrated in FIG. 1), which creates an outwardly emanating pressure wave throughout the balloon fluid 132 that impacts the balloon 204. The impact to the balloon 204 causes the balloon to forcefully disrupt and/or fracture the vascular lesion, such as a calcified vascular lesion, at the treatment site 106.

It is appreciated that by positioning the plasma target 988 away from the guide distal end 722D of the energy guide 722A, damage to the energy guide 722A from the plasma bubble 134 is less likely to occur than if the plasma bubble 134 was generated at or more proximate the guide distal end 722D of the energy guide 722A. Stated another way, the presence of the plasma target 988, and positioning the plasma target 988 away from the guide distal end 722D of the energy guide 722A, causes the plasma bubble 134 to in turn be generated away from the guide distal end 722D of the energy guide 722A, reducing the likelihood of damage to the energy guide 722A.

It is further appreciated that the plasma target ring 982 can include any suitable number of plasma targets 988. For example, in various embodiments, the plasma target ring 982 can be configured to include as many plasma rings 988 as there are energy guides 722A included and/or utilized within the respective treatment device 243. In other embodiments, the plasma target ring 982 can be configured to include as many plasma rings 988 as there are grooves included within the guide positioner 780, e.g., up to six in the embodiments illustrated in the Figures.

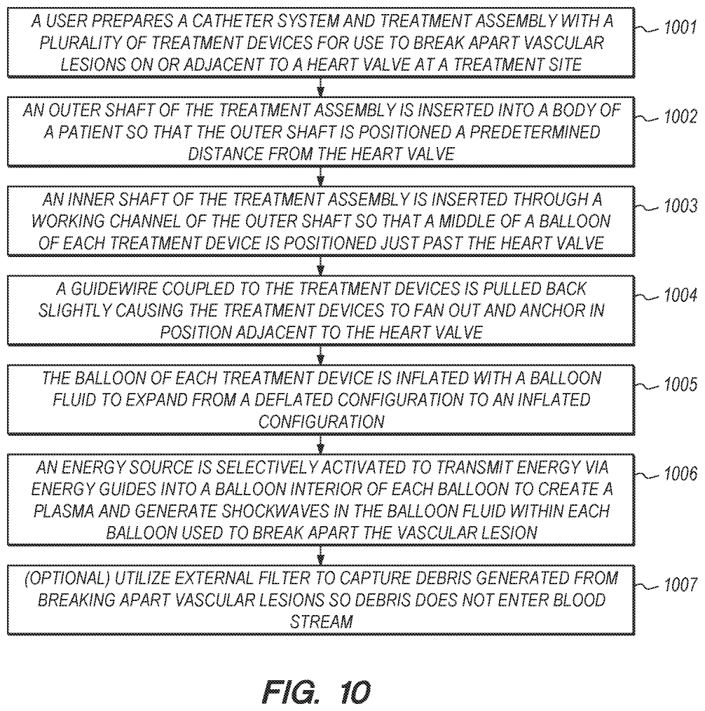

FIG. 10 is a flowchart that illustrates one representative application of a use of the valvuloplasty treatment system as part of the catheter system. More particularly, FIG. 10 illustrates one representative application of the valvuloplasty treatment system for breaking up vascular lesions, such as calcified vascular lesions, adjacent to the valve wall and/or between adjacent leaflets within the tricuspid valve.

It is recognized that in nonexclusive alternative embodiments, the method can include additional steps other than those specifically delineated herein or can omit certain of the steps that are specifically delineated herein. Moreover, in some embodiments, the order of the steps described below can be modified without deviating from the spirit of the present invention.

At step 1001, a user or operator prepares the catheter system for use in order to break apart one or more vascular lesions, such as calcified vascular lesions, adjacent to a valve wall and/or on or between adjacent leaflets within a heart valve at a treatment site. In particular, the user or operator can couple an energy guide bundle including a plurality of energy guides to a system console, and thus to an appropriate energy source. The user or operator can also operatively couple a valvuloplasty treatment system (“treatment system”), such as described in detail herein, to a source manifold of the catheter system.

At step 1002, a multi-lumen outer shaft (“outer shaft”) of the treatment system is inserted into a body of a patient via an artery, such as the femoral artery in the groin area, or other suitable blood vessel of the patient, so that the outer shaft is positioned a predetermined distance, e.g., 10-15 millimeters, away from the heart valve.

At step 1003, a movable multi-lumen inner shaft (“inner shaft”) of the treatment system, with a plurality of spaced apart, individual treatment devices coupled thereto and with a guidewire extending therethrough, is inserted through a working channel of the outer shaft such that a middle of a balloon of each of the treatment devices is positioned just past the leaflets of the heart valve. In various implementations, a device distal end of each treatment device is coupled to a deployment collet that is fixedly secured to the guidewire. In certain implementations, during initial insertion of the inner shaft, the individual treatment devices can be coupled to the inner shaft in a first (retracted) position, with the balloon positioned substantially directly adjacent to the inner shaft. Subsequently, in some such implementations, the treatment devices can be moved to a second (extended) position relative to the inner shaft, with the balloon being spaced apart from the inner shaft.

At step 1004, with the aid of an imaging device such as a CMOS sensor, the guidewire is pulled back slightly, while maintaining the position of the inner shaft and a device proximal end of each of the treatment devices, causing the treatment devices to fan out and to anchor between the leaflets, with the middle of each balloon being positioned substantially adjacent to the treatment site on or adjacent to the leaflets of the heart valve.

At step 1005, the balloon of each of the treatment devices is inflated with a balloon fluid to expand from a deflated configuration to an inflated configuration.

At step 1006, the energy source is selectively activated to transmit energy from the energy source through the plurality of energy guides and into a balloon interior of the balloon of each of the treatment devices. This, in turn, creates a plasma in the balloon fluid within the balloon interior of each of the balloons to generate pressure waves that are used to break up the vascular lesions adjacent to the valve wall and/or on or between adjacent leaflets within the heart valve at the treatment site. It is appreciated that depending upon the particular condition, size and position of the vascular lesions, the treatment system can utilize any number of the individual treatment devices, such as one, two, or three in a treatment system that includes three spaced apart, individual treatment devices, during any given treatment procedure.

At step 1007, an optional external filter can be used to capture and/or trap debris generated from the breaking up of the vascular lesions to inhibit such debris from entering the blood stream.

It should be noted that, as used in this specification and the appended claims, the singular forms “a,” “an,” and “the” include plural referents unless the content and/or context clearly dictates otherwise. It should also be noted that the term “or” is generally employed in its sense including “and/or” unless the content or context clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

The headings used herein are provided for consistency with suggestions under 37 CFR 1.77 or otherwise to provide organizational cues. These headings shall not be viewed to limit or characterize the invention(s) set out in any claims that may issue from this disclosure. As an example, a description of a technology in the “Background” is not an admission that technology is prior art to any invention(s) in this disclosure. Neither is the “Summary” or “Abstract” to be considered as a characterization of the invention(s) set forth in issued claims.

The embodiments described herein are not intended to be exhaustive or to limit the invention to the precise forms disclosed in the present detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

It is understood that although a number of different embodiments of the catheter system and the tissue identification system have been illustrated and described herein, one or more features of any one embodiment can be combined with one or more features of one or more of the other embodiments, provided that such combination satisfies the intent of the present invention.

While a number of exemplary aspects and embodiments of the catheter system and the tissue identification system have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope, and no limitations are intended to the details of construction or design herein shown.

What is claimed is:

1. A catheter system for treating a vascular lesion within or adjacent to a heart valve within a body of a patient, the catheter system comprising:
an energy source that generates energy; and
a plurality of spaced apart treatment devices, each treatment device including (i) a balloon that is positionable substantially adjacent to the vascular lesion, the balloon having a balloon wall that defines a balloon interior, the balloon being configured to retain a balloon fluid within the balloon interior; and (ii) at least one of a plurality of energy guides that receive energy from the energy source so that plasma is formed in the balloon fluid within the balloon interior.

2. The catheter system of claim 1 wherein the heart valve includes a valve wall, and the balloon of each of the treatment devices is configured to be positioned adjacent to the valve wall.

3. The catheter system of claim 1 wherein each treatment device further includes an inflation tube, and the balloon fluid is transmitted into the balloon interior via the inflation tube.

4. The catheter system of claim 3 wherein the balloon of each of the treatment devices includes a balloon proximal end that is coupled to the inflation tube.

5. The catheter system of claim 1 further comprising a plurality of plasma generators, with one corresponding plasma generator of the plurality of plasma generators being positioned near a guide distal end of each of the plurality of energy guides, wherein each plasma generator is configured to generate the plasma in the balloon fluid within the balloon interior.

6. The catheter system of claim 1 wherein the plasma formation causes rapid bubble formation and imparts pressure waves upon the balloon wall of each of the balloons adjacent to the vascular lesion.

7. The catheter system of claim 1 wherein the energy source generates pulses of energy that are guided along each of the plurality of energy guides into the balloon interior of each balloon to induce the plasma formation in the balloon fluid within the balloon interior of each of the balloons.

8. The catheter system of claim 1 wherein the energy source is a laser source that provides pulses of laser energy.

9. The catheter system of claim 1 wherein at least one of the plurality of energy guides includes an optical fiber.

10. The catheter system of claim 1 wherein the energy source is a high voltage energy source that provides pulses of high voltage.

11. The catheter system of claim 1 wherein at least one of the plurality of energy guides includes an electrode pair including spaced apart electrodes that extend into the balloon interior; and wherein pulses of high voltage from the energy source are applied to the electrodes and form an electrical arc across the electrodes.

12. The catheter system of claim 1 further comprising an inner shaft, and wherein a device proximal end of each of the plurality of spaced apart treatment devices is coupled to the inner shaft.

13. The catheter system of claim 12 further comprising a plurality of device couplers; and wherein the device proximal end of each of the plurality of spaced apart treatment devices is coupled to the inner shaft via one of the plurality of device couplers.

14. The catheter system of claim 13 wherein each treatment device further includes an inflation tube, the balloon fluid being transmittable into the balloon interior via the inflation tube, the inner shaft including an inner shaft body that defines a plurality of inner shaft lumens, and the inflation tube of the treatment devices each being coupled to one of the plurality of inner shaft lumens.

15. The catheter system of claim 14 further comprising a guidewire that is configured to guide movement of the plurality of treatment devices so that the balloon of each of the treatment devices is positioned substantially adjacent to the vascular lesion, and wherein the catheter system includes three spaced apart treatment devices that are spaced apart approximately 120 degrees from one another about the guidewire.

16. The catheter system of claim 15 further comprising a deployment collet that is fixedly secured to the guidewire such that movement of the guidewire causes corresponding movement of the deployment collet.

17. The catheter system of claim 16 wherein the guidewire is positioned to extend through the heart valve and the inner shaft is configured to be fixed in position relative to the heart valve during use of the catheter system; and wherein pulling back on the guidewire causes the treatment devices to fan outwardly so that the balloon of each treatment device moves toward the vascular lesion.

18. The catheter system of claim 17 wherein a device distal end of each of the treatment devices is coupled to the deployment collet, and each treatment device further includes an inner tube that is coupled to the deployment collet at the device distal end of each of the treatment devices.

19. The catheter system of claim 18 wherein each treatment device further includes a guide positioner that is positioned about the inner tube, the guide positioner being configured to control a position of the at least one of the plurality of energy guides that is included within the treatment device.

20. The catheter system of claim 1 wherein at least one of the balloons includes a drug eluting coating.

21. A method for treating a vascular lesion within or adjacent to a heart valve utilizing the catheter system of claim 1.
